(19) United States
(12) Patent Application Publication
(10) Pub. No.: US 2024/0173039 A1
(43) Pub. Date: May 30, 2024

(54) DETECTION METHOD FOR SURGICAL DRILLING AND END EFFECTOR USED THEREIN

(71) Applicant: METAL INDUSTRIES RESEARCH & DEVELOPMENT CENTRE, Kaohsiung City (TW)

(72) Inventors: Kai-Szu Lo, Tainan City (TW); Ming-Hui Chen, Kaohsiung City (TW); Hsien-Ju Wu, Kaohsiung City (TW)

(21) Appl. No.: 18/070,546

(22) Filed: Nov. 29, 2022

(51) Int. Cl. A61B 17/16; A61B 90/00

(52) U.S. Cl. CPC A61B 17/1615; A61B 90/06; A61B 2017/00057

(57) ABSTRACT

In a detection method for surgical drilling, an end effector mounted on a drilling machine is provided to detect whether a drill bit of the drilling machine will produce an oversized hole or an oval-shaped hole on a predetermined drill area during a drilling procedure. The end effector includes a carrier and at least one optic bundle which is provided to transmit a light to a detection point and transmit a detect light reflected from the detection point to a processor. A distance value calculated by the processor according to the detection light is provided to estimate whether a drilling strategy, such as feed rate, spindle speed and tilt angle of the drill bit, with or without pecking, needs to be adjusted before the drilling procedure.

### FIELD OF THE INVENTION

This invention relates to a detection method for surgical drilling and an end effector used therein, and more particularly to a detection method for surgical drilling and an end effector used to estimate whether a drilling strategy, e.g. feed rate, spindle speed and tilt angle of a drill bit, with or without pecking, has to be adjusted before a drilling procedure.

### BACKGROUND OF THE INVENTION

A bone drilling machine is usually mounted on a robotic arm, such as multi-axes robotic arm, and moved by the robotic arm to dill a predetermined area on a bone. When the predetermined area on the bone is not flat enough (for example, the bone's surface is too curved), patient's respiratory movement is too much, operation table where the patient is laying on or the robotic arm is moved by accidental contact, an oversized hole or an oval-shaped hole, which may mismatch with bone screw or break bone, may be generated by a drill bit of the bone drilling machine during drilling procedure.

### SUMMARY

One object of the present invention is to provide a detection method for surgical drilling and an end effector used therein. The end effector mounted on a drilling machine is provided to estimate whether a drill bit will cause an oversized hole or oval-shaped hole on a predetermined drill area during a drilling procedure, thus the detection method of the present invention can early adjust drilling strategy, e.g. feed rate, spindle speed and tilt angle of the drill bit, with or without pecking.

A detection method for surgical drilling of the present invention is provided to detect whether a drill bit of a drilling machine will generate an oversized hole or an oval-shaped hole on a predetermined drill area during a drilling procedure. In a first step of the detection method, the drilling machine with an end effector is moved to the outside of the predetermined drill area to allow the drill bit of the drilling machine to align with the predetermined drill area and allow a bit end of the drill bit to be located at an initial position. The end effector includes a carrier, a first optic bundle and a second optic bundle, the first and second optic bundles are positioned in the carrier. A first terminal of the first optic bundle is visible from the carrier and projects a first light to a first detection point on the predetermined area. A second terminal of the second optic bundle is visible from the carrier and projects a second light to a second detection point on the predetermined area. In a second step of the detection method, a first detection light reflected from the first detection point is transmitted to a processor through the first optic bundle to generate a first distance value, and a second detection light reflected from the second detection point is transmitted to the processor through the second optic bundle to generate a second distance value. The drilling procedure is able to be started if a first difference value between the first and second distance values is within a predetermined tolerance range, and the drilling procedure is unable to be started if the first difference value is not within the predetermined tolerance range.

An end effector of the present invention is designed to be mounted on a drilling machine and used to detect whether a drill bit will generate an oversized hole or an oval-shaped hole on a predetermined drill area during a drilling procedure. The end effector includes a carrier and at least one optic bundle which is positioned in the carrier. A terminal of the optic bundle is visible from the carrier to allow a light to project to a detection point on a predetermined drill area, and a detection light reflected from the detection point is transmitted to a processor through the optic bundle and used to calculate a distance value between the optic bundle and the detection point. According to the distance value, it is able to estimate whether the drilling procedure is allowed to proceed.

In the present invention, the end effector is provided to detect whether a drilling strategy (e.g. feed rate, spindle speed and tilt angle of the drill bit, with or without pecking) is appreciate for drilling the predetermined drill area before the drilling procedure.

### DESCRIPTION OF THE DRAWINGS

FIG. 1 is a flowchart illustrating a detection method for surgical drilling in accordance with one embodiment of the present invention.

FIG. 2 is a perspective assembly diagram illustrating an end effector mounted on a drilling machine in accordance with one embodiment of the present invention.

FIG. 3 is a perspective exploded diagram illustrating an end effector mounted on a drilling machine in accordance with one embodiment of the present invention.

FIG. 4 is a cross-section view diagram illustrating an end effector mounted on a drilling machine in accordance with one embodiment of the present invention.

FIG. 5 is a cross-section view diagram illustrating an end effector mounted on a drilling machine in accordance with one embodiment of the present invention.

FIG. 6 is a cross-section view diagram illustrating an end effector mounted on a drilling machine in accordance with one embodiment of the present invention.

FIG. 7 is a perspective assembly diagram illustrating an end effector used in a calibration step in accordance with one embodiment of the present invention.

FIG. 8 is a perspective assembly diagram illustrating an end effector used for detecting a predetermined drill area in accordance with one embodiment of the present invention.

FIG. 9 is a cross-section view diagram illustrating an end effector mounted on a drilling machine in accordance with another embodiment of the present invention.

### DETAILED DESCRIPTION OF THE INVENTION

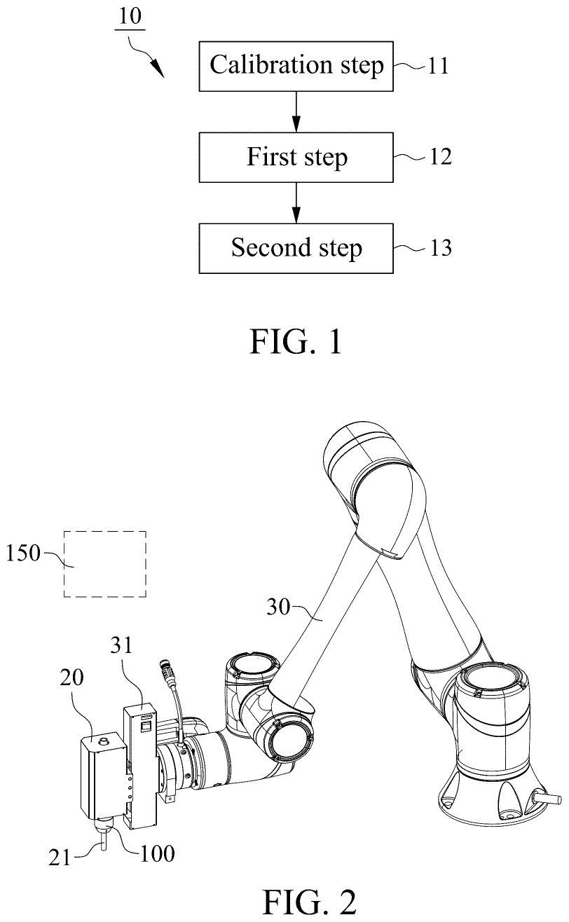

With reference to FIGS. 1, 2 and 8, a detection method 10 for surgical drilling disclosed in the present invention is provided to detect whether a drill bit 21 of a drilling machine 20 will generate an oversized hole or unexpected oval-shaped hole on a predetermined drill area A during a drilling procedure. The detection method 10 includes a first step 12, a second step 13, and preferably, the detection method 10 further includes a calibration step 11.

With reference to FIGS. 1 to 3, an end effector 100 used in the detection method 10 is mounted on the drilling machine 20 and it includes a carrier 110 and at least one optic bundle. In a first embodiment of the present invention, the end effector 100 includes the carrier 110, a first optic bundle 120, a second optic bundle 130 and a third optic bundle 140. The first, second and third optic bundles 120, 130 and 140 are positioned in the carrier 110, a first terminal 121 of the first optic bundle 120, a second terminal 131 of the second optic bundle 130 and a third terminal 141 of the third optic bundle 140 are visible from the carrier 110. The first, second and third optic bundles 120, 130 and 140 are provided to transmit light.

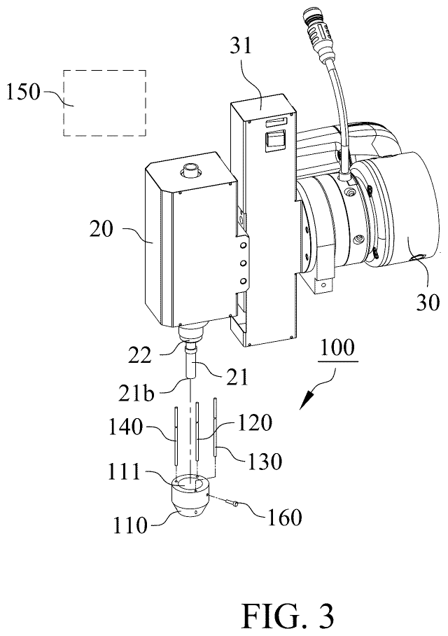

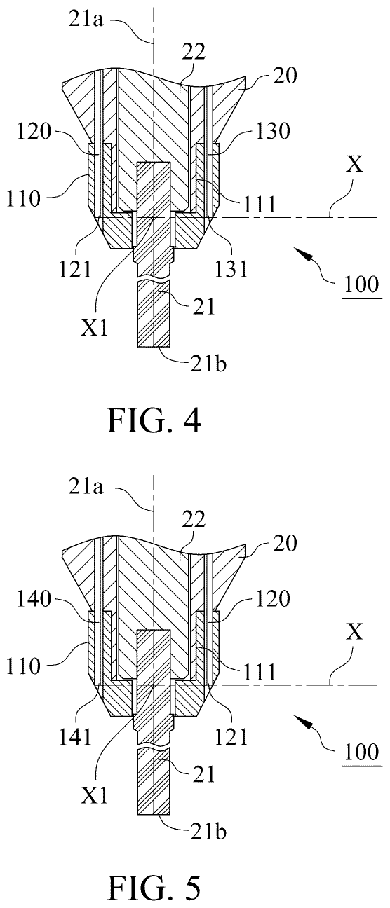

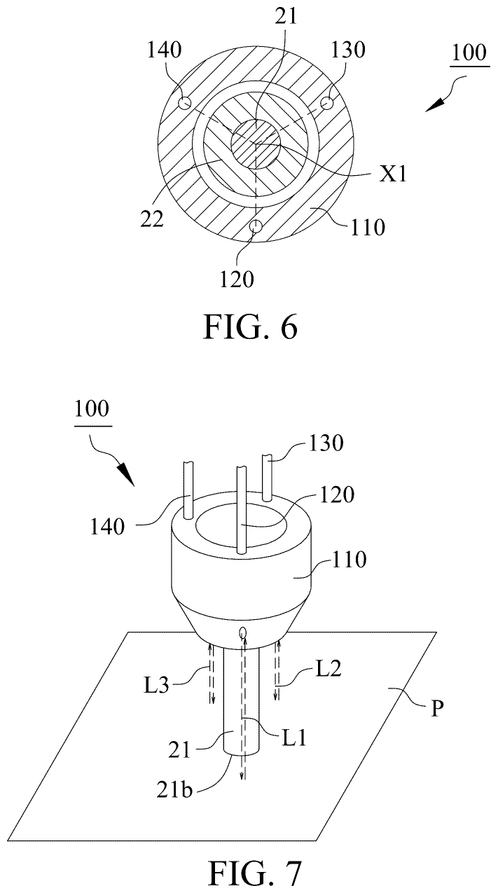

With reference to FIGS. 2, 3 and 8, the drilling machine 20 is mounted on a robotic arm 30, such as multi-axes robotic arm, and in the first embodiment, the drilling machine 20 is mounted on a linear actuator 31 of the robotic arm 30, the linear actuator 31 is used to move the drilling machine 20 toward the predetermined drill area A. Referring to FIGS. 3 to 6, the drilling machine 20 further includes a clamper 22 which is provided to clamp and rotate the drill bit 21. The carrier 110 of the end effector 100 is mounted on the drilling machine 20, and in the first embodiment, there is an accommodation hole 111 provided on the carrier 110, and because of the accommodation hole 111, the carrier 110 can be sleeved outside the clamper 22 of the drilling machine 20 to be mounted on the drilling machine 20. The clamper 22 is rotatably accommodated in the accommodation hole 111, and a bit end 21b of the drill bit 21 is visible from the end effected 100. In the first embodiment, the first, second and third optic bundles 120, 130 and 140 positioned in the carrier 110 are arranged around a centroid which is located at an intersection X1 of a central axis 21a of the drill bit 21 and a horizontal axis X. With reference to FIG. 3, the end effector 100 further includes a fixing component 160 which is provided to allow the carrier 110 to be fixed on the drilling machine 20 and allow the end effector 110 to not rotate with the clamper 22. The fixing component 160 may be screw rod, dowel pin or screw thread which is formed in the accommodation hole 111 and provided to allow the end effector 100 to be screwed on the drilling machine 20.

With reference to FIGS. 3 to 5, in the first embodiment, each of the first, second and third optic bundles 120, 130 and 140 includes at least one first optic fiber (not shown) connected to a light source (not shown, e.g. laser source) and includes at least one second optic fiber (not shown) connected to a light sensing module (not shown, e.g. distance sensor). With reference to FIGS. 2, 3 and 7, in the calibration step 11, the robotic arm 30 moves the drilling machine 20 with the end effector 100 toward a calibration surface P and allows the bit end 21b of the drill bit 21 to contact the calibration surface P, next, the first optic fibers of the first, second and third optic bundles 120, 130 and 140 transmit a first light L1, a second light L2 and a third light L3 from the light source to the calibration surface P respectively, and the second optic fibers of the first, second and third optic bundles 120, 130 and 140 transmit reflected lights from the calibration surface P to a processor 150. Preferably, travel paths of the first light L1, the second light L2 and the third light L3 are parallel to the central axis 21a of the drill bit 21. In the first embodiment, the processor 150 includes, but not limit to, a distance sensor which is provided to measure a distance value between the first optic bundle 120 and the calibration surface P, a distance value between the second optic bundle 130 and the calibration surface P, and a distance value between the third optic bundle 140 and the calibration surface P. While the distance values are not the same, they are adjusted to be the same value in the calibration step 11. Preferably, the calibrated distance values are zero.

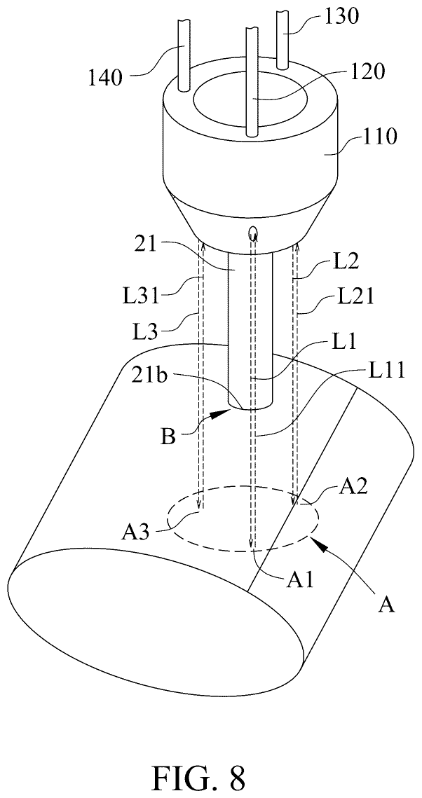

With reference to FIGS. 2 and 8, in the first step 12, the drilling machine 20 with the end effector 100 is moved to outside the predetermined drilling area A (for example, the outside of a bone) to allow the drill bit 21 of the drilling machine 20 to be aligned with the predetermined drill area A and allow the bit end 21b of the drill bit 21 to be located at an initial position B.

With reference to FIGS. 2, 4, 5 and 8, the first light L1, the second light L2 and the third light L3 can be transmitted to the first terminal 121 of the first optic bundle 120, the second terminal 131 of the second optic bundle 130 and the third terminal 141 of the third optic bundle 140, respectively, by the first optic fibers of the first, second and third bundles 120, 130 and 140. The first light L1 projects to a first detection point A1 on the predetermined drill area A from the first terminal 121 of the first optic bundle 120, and a first detection light L11 is reflected from the first detection point A1. The second light L2 projects to a second detection point A2 on the predetermined drill area A from the second terminal 131 of the second optic bundle 130, and a second detection light L21 is reflected from the second detection point A2. The third light L3 projects to a third detection point A3 on the predetermined drill area A from the third terminal 141 of the third optic bundle 140, and a third detection light L31 is reflected from the third detection point A3.

With reference to FIGS. 2, 4, 5 and 8, in the second step 13, the processor 150 receives the first detection light L11 transmitted by the second optic fiber of the first optic bundle 120 to generate a first distance value between the first optic bundle 120 and the predetermined area A, receives the second detection light L21 transmitted by the second optic fiber of the second optic bundle 130 to generate a second distance value between the second optic bundle 130 and the predetermined area A, and receives the third detection light L31 transmitted by the second optic fiber of the third optic bundle 140 to generate a third distance value between the third optic bundle 140 and the predetermined area A. In the first embodiment, the processor 150 further includes, but not limit to, a compute module which is provided to calculate a first difference value between the first and second distance values, calculate a second difference value between the first and third distance values, and calculate a third difference value between the second and third distance values. When the first, second and third difference values are within a predetermined tolerance range, the processor 150 estimates a drilling strategy of the predetermined drill area A, such as feed rate, spindle speed and tilt angle of the drill bit 21, and with or without pecking, is appropriate and the drilling procedure is allowable. On the other hand, while at least one of the first, second and third difference values is not within the predetermined tolerance range, the processor 150 estimates the drilling strategy of the predetermined drill area A is improper and stops to proceed the drilling procedure, thus it is possible to avoid the drill bit 21 from forming a drill hole that is oversized or oval-shaped after the drilling procedure.

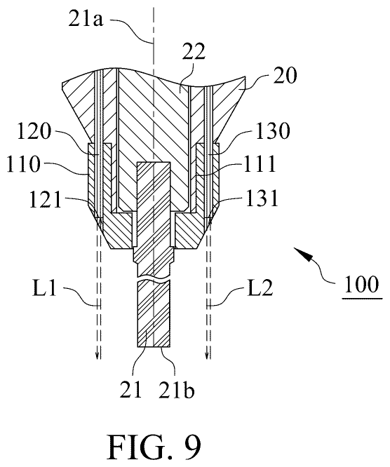

A second embodiment of the present invention is shown in FIG. 9. Different to the first embodiment, the end effector 100 of the second embodiment includes only the first optic bundle 120 and the second optic bundle 130, without the third optic bundle 140. The drill bit 21 is positioned between the first light L1 and the second light L2, and the first terminal 121 of the first optic bundle 120 and the second terminal 131 of the second optic bundle 130 are arranged outside the drill bit 21 and symmetrically arranged with respect to the central axis 21a of the drill bit 21. In the second step, the drilling procedure is allowed to execute as the first difference value between the first and second distance values is within the predetermined tolerance range, and the drilling procedure is not allowed to execute if the first difference value is not within the predetermined tolerance range.

While this invention has been particularly illustrated and described in detail with respect to the preferred embodiments thereof, it will be clearly understood by those skilled in the art that is not limited to the specific features shown and described and various modified and changed in form and details may be made without departing from the scope of the claims.

What is claimed is:

1. A detection method for surgical drilling configured to detect whether a drill bit of a drilling machine will generate an oversized hole or oval-shaped hole on a predetermined drill area during a drilling procedure, the detection method comprising the steps of:
a first step, moving the drilling machine with an end effector to outside of the predetermined drill area, aligning the drill bit of the drilling machine with the predetermined drill area and positioning a bit end of the drill bit at an initial position, wherein the end effector includes a carrier, a first optic bundle and a second optic bundle, the first and second optic bundles are positioned in the carrier, a first terminal of the first optic bundle is visible from the carrier and configured to project a first light to a first detection point on the predetermined drill area, a first detection light is reflected from the first detection point, and a second terminal of the second optic bundle is visible from the carrier and configured to project a second light to a second detection point on the predetermined drill area, a second detection light is reflected from the second detection point; and
a second step, transmitting the first detection light to a processor through the first optic bundle to obtain a first distance value and transmitting the second detection light to the processor through the second optic bundle to obtain a second distance value, wherein the drilling procedure is allowed to execute while a first difference value between the first and second distance values is within a predetermined tolerance range, and the drilling procedure is not allowed to execute while the first difference value is not within the predetermined tolerance range.

2. The detection method for surgical drilling in accordance with claim 1, wherein the end effector further includes a third optic bundle which is positioned in the carrier, a third terminal of the third optic bundle is visible from the carrier and configured to project a third light to a third detection point on the predetermined drill area and a third detection is reflected from the third detection point in the first step, the third detection light is transmitted to the processor through the third optic bundle to obtain a third distance value in the second step, the drilling procedure is allowed to execute while a second difference value between the first and third distance values and a third difference value between the second and third distance values are within the predetermined tolerance range, and the drilling procedure is not allowed to execute while the second or third difference value is not within the predetermined tolerance range.

3. The detection method for surgical drilling in accordance with claim 2, wherein the first, second and third optic bundles positioned in the carrier are arranged around a centroid which is an intersection of a central axis of the drill bit and a horizontal axis.

4. The detection method for surgical drilling in accordance with claim 1, wherein travel paths of the first light and the second light are parallel to a central axis of the drill bit.

5. The detection method for surgical drilling in accordance with claim 1, wherein the drilling machine includes a clamper configured to clamp and rotate the drill bit, the carrier of the end effector is mounted on the drilling machine, the clamper is accommodated in an accommodation hole of the carrier, the bit end of the drill bit is visible from the end effector, and the end effector is configured to not be rotated with the clamper.

6. The detection method for surgical drilling in accordance with claim 1, wherein the drill bit is located between the first light and the second light.

7. The detection method for surgical drilling in accordance with claim 6, wherein the first terminal of the first optic bundle and the second terminal of the second optic bundle are arranged outside the drill bit and symmetrically arranged with respect to a central axis of the drill bit.

8. An end effector configured to be mounted on a drilling machine and detect whether a drill bit will generate an oversized hole or oval-shaped hole on a predetermined drill area during a drilling procedure, the end effector comprising:
a carrier; and
at least one optic bundle positioned in the carrier and including a terminal visible from the carrier, the at least one optic bundle is configured to project a light to a detection point on the predetermined drill area and transmit a detection light reflected from the detection point to a processor to obtain a distance value, and the distance value is configured to estimate whether the drilling procedure is allowed to execute.

9. The end effector in accordance with claim 8 comprising a plurality of optic bundles, wherein the plurality of optic bundles are positioned in the carrier and arranged around a centroid which is an intersection of a central axis of the drill bit and a horizontal axis.

10. The end effector in accordance with claim 8 comprising two optic bundles, wherein the two optic bundles are positioned to allow the drill bit to be located between a light projecting from one of the two optic bundles and a light projecting from the other one of the two optic bundles.

11. The end effector in accordance with claim 10, wherein terminals of the two optic bundles are configured to be arranged outside the drill bit and symmetrically arranged with respect to a central axis of the drill bit.

12. The end effector in accordance with claim 8, wherein an accommodation hole on the carrier is configured to accommodate a clamper which is configured to clamp the drill bit, the end effector is configured to not cover the bit end of the drill bit and configured to not be rotated with the clamper.

13. The end effector in accordance with claim 8 further comprising a fixing component, wherein the carrier is mounted on the drilling machine by the fixing component.
